(19) United States
(12) Patent Application Publication
(10) Pub. No.: US 2025/0152056 A1
(43) Pub. Date: May 15, 2025

(54) Device for Processing Signals Indicative of Health

(71) Applicant: VitalTrace Pty Ltd, Wembley (AU)

(72) Inventors: Toby Angus Digney, Bassendean (AU); Henry Jian Chen, Balga (AU); Michael Tom Challenor, Maylands (AU); Arjun Siddharth Kaushik, City Beach (AU)

(21) Appl. No.: 18/836,182

(22) PCT Filed: Feb. 14, 2023
(86) PCT No.: PCT/AU2023/050097

§ 371 Date: Aug. 6, 2024

(30) Foreign Application Priority Data
Feb. 14, 2022 (AU) 2022900307

(51) Int. Cl. A61B 5/1482; A61B 5/145; A61B 5/344

(52) U.S. Cl. CPC A61B 5/1482; A61B 5/14546; A61B 5/344

(57) ABSTRACT

A device for processing signals indicative of health in a subject, the device comprising circuitry operable to receive input comprising a first signal indicative of health and a signal indicative of health process the first signal indicative of health process the second signal indicative of health and provide an output arising from the processing of the first signal indicative of health and the second signal indicative of health.

### TECHNICAL FIELD

The present invention relates to a device for processing signals indicative of health.

### BACKGROUND ART

During labour and delivery, there is a risk of damage to the fetal brain, neurological systems and other end organs as a result of hypoxia, otherwise known as fetal asphyxia. Fetal asphyxia can cause long term, debilitating sequelae for the baby and their family.

Currently, clinicians monitor uterine contractions and fetal heart rate (FHR) or electrophysiological signals, such as ECG, using a cardiotocograph (CTG), wherein a change in signal patterns may indicate fetal asphyxia. However, changes in signal patterns are typically only assessed subjectively, which has led to a high number of false positives. This subjectivity can also lead to delayed or missed diagnoses, resulting in negative health outcomes. Consequently, many caesarean sections are performed based upon false indications of fetal distress as a defensive monitor to reduce the risk of intrapartum fetal asphyxia. Due to the large number of false positives, the rate of non-elective caesarean sections has increased. Caesarean sections come with a number of problems. For example, caesarean sections are associated with increased morbidity for the mother, longer recovery times, and higher rates of post-partum infections. Furthermore, caesarean sections are higher in cost than vaginal deliveries.

Methods exist for fetal distress monitoring, such as fetal lactate monitoring using blood sampling. Lactate is widely considered the gold standard for measuring fetal distress; however, it is difficult to do in standard practice. Lactate can only be assessed by drawing the blood of the fetus during birth and analyzing the blood sample externally. This causes significant distress for the mother and produces a result with a significant time delay of 20-30 mins. As such, despite its potential benefits as a fetal monitoring method, it is rarely used.

Electrophysiology is the study of the electrical properties of biological cells and tissue, and generally involves obtaining and processing measurements of changes in voltage or electrical current in biological tissue or in entire organs such as the heart. Electrophysiology techniques involve placing ECG sensor electrodes at target regions on an individual's skin to capture an electrophysiological signal, such as an ECG waveform. This waveform provides both the heart rate of a subject but also information about electrical pulses from the heart, whose magnitude and direction can be indicative of health conditions.

A typical ECG requires at least three electrodes, termed here the ECG reference electrode (ERE), ECG working electrode (EWE), and an ECG ground electrode (EGE). The three electrode ECG set-up enables the maternal heartbeat to be filtered from the fetal heartbeat. This is critical as the electrical pulse caused by the maternal heartbeat is stronger than the electrical pulse caused by the fetal heartbeat. This arrangement is described in U.S. Pat. No. 5,062,426.

Previous systems have attempted to use various continuous monitoring technologies, such as in US20170112428 which used a micro-dialysis technique to sample extracellular fluid to determine analyte concentrations like lactate. However, this results in significant lag between the result and sampling due to the micro-dialysis process, which involves pumping a dialysate solution via microcapillary tubes to an external analysis unit.

Another method to monitor fetal health during delivery is provided in WO2003088837, which combines a temperature probe with an ECG. However, whilst this allows for continuous monitoring of temperature, this parameter is prone to interference given the proximity of the mother and there is no strong evidence of any correlation between temperature and fetal asphyxia.

Numerous systems have been developed for wearable electronic sensing units for use in monitoring exercise, such as US20160157779. However, none have combined these functions in a fetal monitoring context, which requires consideration for the unique difficulties of childbirth.

Fetal scalp electrode systems and connector devices have been described in the prior art, such as U.S. Pat. No. 6,363,272, where a wearable leg-plate connector assembly is described. This device does not contain a printed circuit board (PCB) assembly or any electronics and simply provides a connector system to be used in conjunction with a wired cable to transmit electrical signals from the mother and baby to a monitor. U.S. Pat. No. 5,168,876 describes a leg plate as a connector device which contains a PCB, but this PCB only contains tracers which electrically connect input contacts and the ground electrode with respective output leads which connect to a fetal monitor.

There is a need to monitor the concentration of an analyte indicative of fetal asphyxia continuously and in real-time, or at least provide an alternative or compliment to conventional devices and methods. The present invention, in embodiments, seeks to provide the ability to process a signal indicative of concentration of an analyte in a fetus and an electrophysiological signal of the fetus, preferably in a way which is in a compact form for a mother to comfortably wear.

Alternatively, a device that provides the ability to process a signal indicative of concentration of an analyte and an electrophysiological signal may be used in variety of applications, such as exercise monitoring, horse racing, and health monitoring more broadly. Lactate is generated from exercise when the demand for ATP and oxygen exceeds supply, as occurs during intense exercise. In such situations, the working muscles generate energy anaerobically and lactate can accumulate to high levels. Real-time monitoring of exercise induced lactate production would assist trainers, athletes etc with training and health assessment. Lactate monitoring can also be used to monitor severe health conditions such as sepsis and has many clinical applications both in humans and animals.

The previous discussion of the background art is intended to facilitate an understanding of the present invention only. The discussion is not an acknowledgement or admission that any of the material referred to is or was part of the common general knowledge as at the priority date of the application.

### SUMMARY OF THE INVENTION

According to a first broad aspect of the present invention, there is provided a device for processing signals indicative of health, the device comprising circuitry operable to:
receive input comprising a first signal indicative of health and a second signal indicative of health;
process the first signal indicative of health;
process the second signal indicative of health; and
provide an output arising from the processing of the first signal indicative of health and the second signal indicative of health.

Preferably, the first signal indicative of health comprises an electroanalytical signal indicative of concentration of an analyte in a subject. The subject may be a fetus. Alternatively, the subject may be an athlete, animal, human, child, etc as the first signal indicative of health may be used in variety of applications, such as exercise monitoring, horse racing, and health monitoring.

Preferably, the second signal indicative of health comprises an electrophysiological signal of the subject, for example the fetus.

Preferably, the circuitry comprises at least two circuits, the first circuit being an electroanalytical signal processing circuit adapted to process the electroanalytical signal indicative of the concentration of the analyte in the subject, and the second circuit being an electrophysiological signal processing circuit adapted to process the electrophysiological signal of the subject.

Preferably, the circuitry is provided, on or otherwise implemented in, a printed circuit board (PCB)/printed circuit board assembly (PCBA).

Preferably, the processing comprises at least one of: amplification; conversion from analogue to digital.

Preferably, the device is provided, on or otherwise implemented in, a unit, to thereby provide an electronic processing unit for processing an electroanalytical signal indicative of the concentration of an analyte in a subject and an electrophysiological signal of the subject.

Preferably the subject is a fetus. Alternatively, the subject is a subject who is exercising, such as a human or a horse. Alternatively, the subject may be a human adult or child.

Preferably, the electroanalytical signal processing circuit and the electrophysiological signal processing circuit share a common reference voltage. Preferably, the common reference voltage between the electrophysiological signal processing circuit and the electroanalytical signal processing circuit is in the form of a connection, such as an electrical wire on the PCB/PCBA, connecting a first biosensor amplifier component to a second electrophysiology amplifier component which also shares an electrical connection with a biosensor reference electrode and an electrocardiogram electrode, preferably the electrocardiogram working electrode (EWE).

Optionally, the device may further comprise one or more of the following:
i. an energy source (for example a battery or wired connection);
ii. a wired transmission means to transmit an electrophysiological signal, preferably an analogue electrophysiological signal, and/or an electroanalytical signal, preferably an analogue electroanalytical signal, from a biosensor device to the device; and/or
iii. a communication connection (wired or wireless) for the transfer of a processed electrophysiological signal, preferably an amplified and/or digital electrophysiological signal, and/or a processed electroanalytical signal, preferably an amplified and/or digital electroanalytical signal, to an external computing device.

Optionally, the device may further comprise one or more of the following:
i. an accelerometer for the detection of maternal movement and/or the movement of a biosensor device;
ii. connection studs;
iii. an adhesive patch, strap, or bandage for securing the electronic processing unit to the subject if the subject is not a fetus (for example, the subject is exercising or is in need of health monitoring);
iv. an adhesive patch, strap, or bandage for securing the electronic processing unit to a mother of a fetus;
v. a top cover; and/or
vi. a bottom cover.

According to a second broad aspect of the present invention, there is provided a system for processing an electroanalytical signal indicative of the concentration of an analyte in a subject and an electrophysiological signal of the subject, the system comprising:
a) a sensor device attachable to a subject, said sensor device comprising (i) a biosensor device capable of and operable for sensing the concentration of the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of the subject;
b) a transmission means to transmit the electroanalytical signal and the electrophysiological signal to a device for processing signals indicative of health; and
c) any embodiment of a device for processing signals indicative of health according to the first broad aspect of the present invention, or as herein described.

According to a third broad aspect of the present invention, there is provided a system for processing an electroanalytical signal indicative of the concentration of an analyte in a fetus and an electrophysiological signal of the fetus, the system comprising:
a) a fetal sensor device attachable to the fetus, said fetal sensor device comprising (i) a biosensor device capable of and operable for sensing the concentration of the analyte in the fetus and (ii) an electrocardiogram (ECG) capable of and operable for sensing an ECG waveform of the fetus, wherein said biosensor device of the fetal sensor device generates an electroanalytical signal indicative of the concentration of the analyte in the fetus and said ECG of the fetal sensor device generates an electrophysiological signal of the fetus;
b) a transmission means to transmit the electroanalytical signal and the electrophysiological signal to a device for processing signals indicative of health; and
c) any embodiment of a device for processing signals indicative of health according to the first broad aspect of the present invention, or as herein described.

According to a fourth broad aspect of the present invention, there is provided a method for processing an electroanalytical signal indicative of the concentration of an analyte in a subject and an electrophysiological signal of the subject, the method comprising the steps of:
a) attaching a sensor device to the subject, said sensor device comprising (i) a biosensor device capable of and operable for sensing the concentration of the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of the subject;
b) transmitting the electroanalytical signal and the electrophysiological signal to a device for processing signals indicative of health via a transmission means; and processing the electroanalytical signal and the electrophysiological signal in the device for processing signals indicative of health, wherein the device for processing signals indicative of health comprises any embodiment of a device for processing signals indicative of health according to the first broad aspect of the present invention, or as herein described.

According to a fifth broad aspect of the present invention, there is provided a method for processing an electroanalytical signal indicative of the concentration of an analyte in a fetus and an electrophysiological signal of the fetus, the method comprising the steps of:
a) attaching a fetal sensor device to the fetus, said fetal sensor device comprising (i) a biosensor device capable of and operable for sensing the concentration of the analyte in the fetus and (ii) an electrocardiogram (ECG) capable of and operable for sensing an ECG waveform of the fetus, wherein said biosensor device of the fetal sensor device generates an electroanalytical signal indicative of the concentration of the analyte in the fetus and said ECG of the fetal sensor device generates an electrophysiological signal of a fetus;
b) transmitting the electroanalytical signal and the electrophysiological signal to a device for processing signals indicative of health via a transmission means; and processing the electroanalytical signal and the electrophysiological signal in the device for processing signals indicative of health, wherein the device for processing signals indicative of health comprises any embodiment of a device for processing signals indicative of health according to the first broad aspect of the present invention, or as herein described.

### BRIEF DESCRIPTIONS OF THE DRAWINGS

FIG. 1 is a schematic representation of a system for the simultaneous processing of an electroanalytical signal indicative of the concentration of an analyte in a fetus and an electrophysiological signal indicative of the ECG waveform of the fetus.

FIG. 2 is a schematic view of an example (the first arrangement) of a printed circuit board (PCB) of the wearable external electronic processing unit of an embodiment of the present invention.

FIG. 3 is a schematic view of an example (the second arrangement) of a PCB of the wearable external electronic processing unit of an embodiment of the present invention.

FIG. 4 is a schematic view of an example (the third arrangement) of a PCB of the wearable external electronic processing unit of an embodiment of the present invention.

FIG. 5 is a schematic view of an example (the fourth arrangement) of a PCB of the wearable external electronic processing unit of an embodiment of the present invention.

FIG. 6 is a schematic view of an example (the fifth arrangement) of a PCB of the wearable external electronic processing unit of an embodiment of the present invention.

FIG. 7 is a schematic view of an example (the sixth arrangement) of a PCB of the wearable external electronic processing unit of an embodiment of the present invention.

FIG. 8 is a schematic expanded view of an embodiment of the electronic processing unit for the simultaneous processing of an electroanalytical signal indicative of the concentration of an analyte in a fetus and an electrophysiological signal indicative of the ECG waveform of the fetus in accordance with an aspect of the present invention.

FIG. 9 is a schematic view of an embodiment of the electronic processing unit for the simultaneous processing of an electroanalytical signal indicative of the concentration of an analyte in a fetus and an electrophysiological signal indicative of the ECG waveform of the fetus in accordance with an aspect of the present invention.

FIG. 10 is a schematic view of an external computing device and the electronic processing unit of the embodiment of the invention described. The electronic processing unit is shown in charging mode.

FIG. 11 is a graph of results from a biosensor during additions of analyte; the ECG sensor is being run concurrently.

### DESCRIPTION OF EMBODIMENTS OF THE INVENTION

Notwithstanding any other forms which may fall within the scope of the present invention, in order that the invention may be more fully understood and put into practice preferred embodiments of the invention will now be described.

False positive indications of fetal distress detected by the conventional method of monitoring a fetus through heart rate monitoring alone may result in an increase in caesarean sections to reduce the time of the delivery.

A device that could combine the ability to monitor fetal electrophysiological signals and lactate continuously would provide clinicians with access to a technique that would be superior to the current standard of care.

The present invention seeks to provide such a device. In one implementation, the device has the form of an electronic processing unit operable to enable simultaneous or near simultaneous processing of an electroanalytical signal indicative of concentration of an analyte in a fetus and an electrophysiological signal of the fetus, preferably using a single common reference voltage. This would advantageously allow the use of a fetal sensor device with fewer cables, and therefore fewer invasive electrodes, improving comfort for both the mother and fetus whilst providing accurate fetal monitoring.

Embodiments of the present invention allow for simultaneous, or near simultaneous, consideration of both signals on one device. This is particularly useful in assessing instances in which the fetal heart rate is non-reassuring, suggesting fetal hypoxia or asphyxia may be occurring. In such cases, it would be advantageous to have a second, confirmatory signal indicative of the state of the fetus. In these cases, measurement of the concentration of an analyte, being a more specific and sensitive measure of fetal asphyxia, would complement the electrophysiological signal. This combination of signals may potentially prevent unnecessary intervention that may occur if the electrophysiological signal was considered in isolation.

Embodiments of the present invention seek to provide a device operable to potentially reduce the invasiveness and complexity of monitoring for fetal distress. Embodiments of the invention provide for the continuous monitoring of one or more analytes, such as lactate or oxygen, and simultaneous, or near simultaneous, monitoring of the fetal heart. This would allow relevant medical professionals, such as a doctor or midwife, to be provided with an indication to act immediately once lactate or heart rate enters a dangerous range.

The device is also applicable to use in a subject who is exercising, such as a human exercising or in the field of horse racing. Lactate is generated from exercise when the demand for ATP and oxygen exceeds supply, as occurs during intense exercise. In such situations, the working muscles generate energy anaerobically during which time lactate can accumulate to high levels. The present device would be able to monitor the lactate levels of a subject in real time, with minimal invasiveness. Therefore, subjects such as runners and racing horses can be monitored as they exercise, rather than merely at the start and end of races or other periods of exercise.

The device is also applicable to health monitoring more broadly. Numerous health conditions such as sepsis could benefit from continuous lactate monitoring. This could be applied to both human adults, human children, and other animals.

### Device

The present invention provides a device for processing signals indicative of health, the device comprising circuitry operable to:
receive input comprising a first signal indicative of health and a second signal indicative of health;
process the first signal indicative of health;
process the second signal indicative of health; and
provide an output arising from the processing of the first signal indicative of health and the second signal indicative of health.

Preferably, the first signal indicative of health comprises an electroanalytical signal indicative of concentration of an analyte in a subject. The subject may be a fetus. Alternatively, the subject may be an athlete, animal, human, child, etc as the first signal indicative of health may be used in variety of applications, such as exercise monitoring, horse racing, and health monitoring.

Preferably, the second signal indicative of health comprises an electrophysiological signal of the subject, for example the fetus.

Preferably, the circuitry comprises at least two circuits, the first circuit being an electroanalytical signal processing circuit adapted to process the electroanalytical signal indicative of the concentration of the analyte in the subject, and the second circuit being an electrophysiological signal processing circuit adapted to process the electrophysiological signal of the subject.

Preferably, the circuitry is provided on, or otherwise implemented in, a printed circuit board (PCB)/printed circuit board assembly (PCBA).

Optionally, the circuitry is provided on, or otherwise implemented in, a flexible printed circuit board (PCB)/printed circuit board assembly (PCBA). A flexible circuit board may comprise a rigid-flexible circuit board, a flexible circuit board, or a series of rigid circuit boards connected by flexible components.

Optionally, the circuitry is provided, or otherwise implemented such that a second circuitry, which may be in the form of a daughter board may operably connect to the printed circuit board (PCB). Optionally, the circuitry is provided, or otherwise implemented such that a plurality of daughter boards may connect to the printed circuit board (PCB).

Preferably, the processing comprises at least one of: amplification; conversion from analogue to digital.

Preferably, the device implements a series of resistors in the electroanalytical and the electrophysiological circuit to provide protection from electrical failure in the device, preventing or mitigating electrical shock. These resistors may be placed anywhere in series to these circuits to provide protection to the subject or if the subject is a fetus, the fetus and the mother.

Preferably, the resistors in the electroanalytical circuit are provided in the form of kelvin connected resistors, which provide protection from electrical failure while also allowing for improved sensor current acquisition.

Preferably, the device is implemented in a unit, to thereby provide an electronic processing unit for processing an electroanalytical signal indicative of the concentration of an analyte in a subject and an electrophysiological signal of the subject.

Preferably, the electroanalytical signal processing circuit and the electrophysiological signal processing circuit share a common reference voltage. Preferably, the common reference voltage between the electrophysiological signal processing circuit and the electroanalytical signal processing circuit is in the form of a connection, such as an electrical wire on the PCB/PCBA, connecting a first biosensor amplifier component to a second electrophysiology amplifier component which also shares an electrical connection with a biosensor reference electrode and an electrocardiogram electrode, preferably the electrocardiogram working electrode (EWE).

Optionally, the device may further comprise one or more of the following:
i. an energy source (for example, a battery or wired connection);
ii. a wired transmission means to transmit an electrophysiological signal, preferably an analogue electrophysiological signal, and/or an electroanalytical signal, preferably an analogue electroanalytical signal, from a fetal sensor device to the device; and/or
iii. a communication connection (wired or wireless) for the transfer of a processed electrophysiological signal, preferably an amplified and/or digital electrophysiological signal, and/or a processed electroanalytical signal, preferably an amplified and/or digital electroanalytical signal, to an external computing device.

Optionally, the device may further comprise one or more of the following:
i. an accelerometer for the detection of maternal movement and/or the movement of a sensor device;
ii. connection studs;
iii. an adhesive patch, strap, or bandage for securing the electronic processing unit to the subject if the subject is not a fetus (for example the subject is exercising);
iv. an adhesive patch or a strap or bandage for securing the electronic processing unit to the mother of a fetus;
v. a top cover; and/or
vi. a bottom cover.

In one embodiment, the device may be flexible, allowing for a more comfortable fit to the subject, including a mother of a fetus if the subject is a fetus. In another embodiment, the circuitry may be flexible to improve the flexibility and comfort of the device.

### Printed Circuit Board/Printed Circuit Board Assembly

In one embodiment, the printed circuit board (PCB)/printed circuit board assembly (PCBA) is designed such that the reference potential for the electrophysiological signal processing circuit that monitors the electrophysiological signal (the electrocardiogram (ECG)) is the same as the reference potential used by the electroanalytical signal processing circuit that monitors the electroanalytical signal indicative of the concentration of the analyte.

In one embodiment, the printed circuit board (PCB)/printed circuit board assembly (PCBA) is designed such that the electrophysiological signal processing circuit that monitors the electrophysiological signal (the electrocardiogram (ECG)) has a reference potential and the electroanalytical signal processing circuit that monitors the electroanalytical signal indicative of the concentration of the analyte has a separate reference potential.

### Electroanalytical Signal and Electrophysiological Signal

The electroanalytical signal indicative of concentration of an analyte of a subject and the electrophysiological signal of the subject are preferably generated using a sensor device. The sensor device preferably comprises a biosensor device and an electrocardiogram (ECG). The sensor device is operably attachable to the subject, for example by insertion into the muscle tissue of the subject.

The electroanalytical signal indicative of concentration of an analyte of a fetus and the electrophysiological signal of the fetus are preferably generated using a fetal sensor device. The fetal sensor device preferably comprises a biosensor device and an electrocardiogram (ECG). The fetal sensor device is operably attachable to the fetus, for example by insertion into the scalp of the fetus.

The analyte to be monitored by the biosensor device of the sensor device (including a fetal sensor device) may be an analyte chosen from the list comprising: lactate, glucose, pyruvate, oxygen, pH, pCO₂, pO₂, pHCO₃, purines, ascorbic acid, uric acid, cholesterol, xanthine, NADPH, cytochrome P450, alcohol, ketones, and others. A person skilled in the art will be aware that numerous analytes may be monitored using biosensor devices. More than one biosensor device may be present in the sensor device. More than one electroanalytical signal indicative of the concentration of an analyte may be monitored using embodiments of the device of the present invention.

Preferably, the device of the present invention comprises an electroanalytical signal processing circuit. This electroanalytical signal processing circuit may detect electroanalytical signals indicative of concentration of an analyte in a subject via chronoamperometry, but one skilled in the art can appreciate that any number of electroanalytical techniques may be chosen from a list comprising: chronoamperometry, chronopotentiometry, cyclic voltammetry, square wave voltammetry, differential pulse voltammetry or other similar techniques. Preferably the signal generated is an analogue signal.

Preferably, the analyte is sensed using a biosensor device attached directly to the subject (including a fetus), said biosensor device being capable of sensing the concentration of the analyte in the subject, wherein said biosensor device generates an electroanalytical signal indicative of the concentration of the analyte in the subject. Preferably, the biosensor device comprises two electrodes for sensing the concentration of an analyte in the subject wherein the first electrode is a biosensor working electrode (BWE), comprising a reactive substance, and the second electrode is a biosensor reference electrode (BRE), and wherein the BWE and BRE are used for sensing the analyte concentration of the subject. The biosensor device may further comprise a biosensor counter electrode (BCE).

Preferably, the device of the present invention comprises an electrophysiological signal processing circuit. This electrophysiological signal processing circuit may detect the electrophysiological signal of the subject, generated using an electrocardiogram (ECG) electrode.

Preferably, the ECG electrode is operably attachable and attached directly to the subject and is operable for and capable of sensing electrophysiological signals such as the ECG waveform of the subject. An ECG generates a complete waveform, with segments that can be interpreted (P, Q, R, S and T waves) contained within it. Heart rate is calculated by determining the time between successive R waves (which correspond to the contraction of the ventricles of the heart). Preferably, the ECG comprises three electrodes, wherein the first electrode is an electrocardiogram working electrode (EWE) connected to the subject, the second electrode is an electrocardiogram reference electrode (ERE) separately attached to the biosensor device, and the third electrode is an electrocardiogram ground electrode (EGE) connected externally to the subject (or the mother if the subject is a fetus). The subject's electrophysiological signals are sensed as the difference in potential between the EWE and ERE. Preferably the signal generated is an analogue signal.

The sensor device comprising a biosensor device and ECG for use with the device of the present invention may be similar to that described in AU2021903268. The sensor device in AU2021903268 is for sensing a fetus, but can easily be adjusted to be implemented in other subjects such as humans or horses for exercise, and for health monitoring.

### Energy Source

The device may optionally comprise an energy source. In one embodiment, the energy source may comprise a wired connection to an external energy source, such as mains electricity or an external computing device.

In one embodiment, the energy source of the electronic processing unit may be provided by a battery. The battery may be provided as a single unit or separated into several batteries. The battery(s) may be single use/disposable, or rechargeable. Preferably the battery uses lithium-ion technology.

### Accelerometer

In one embodiment, the device may include an accelerometer. This would allow the device to be operable to sense the movement of the subject (including a mother and fetus). The signals received from the sensor device attached to the subject may be impacted by movement of the sensor device, and the accelerometer, in an embodiment, would be operable to allow data collected during these periods to be marked, filtered, and classified to reduce the impact of this effect. In this embodiment, the signals received from the accelerometer may be additionally marked on an external computing device. This provides the advantage that the physician, veterinarian or sport physiologist for example, can determine when sudden changes in the analyte or heart rate may be caused by movement of the subject or mother if the subject is a fetus rather than true changes in these parameters.

In one embodiment, the accelerometer may be located on the wire coupling the device to the sensor device or within the sensor. This would enable the accelerometer to more accurately detect movement of the sensor device, which may allow for filtering or marking of data produced during these periods.

The accelerometer may also or alternatively enable the use of ‘gesture’ controls to allow the user to interact with the device. An example could be tapping the device three times to enable a Bluetooth™ pairing with the external computing device.

The accelerometer may also or alternatively be used as an energy or power saving feature. When a continued lack of movement for a predetermined period of time is detected, the device may enter a low-power mode to save power.

### External Connections

The device may be connected to an external computing device and/or a sensor device (including a fetal sensor device).

### Connection to Sensor Device

The device may be connected to a sensor device by a transmission means. The transmission means transmits an electroanalytical signal, preferably an analogue electroanalytical signal, and an electrophysiological signal, preferably an analogue electrophysiological signal, from the sensor device to the electronic processing unit.

Preferably the transmission means comprises sensor wires, and one or more sensor connectors. When the transmission means comprises sensor wires, there is improved reliability and miniaturization as compared to wireless connection technologies. Additionally, it is difficult to transmit low-energy radio waves through the human body as would be necessary in this application.

The sensor connectors may feature a multiple pin design. In one example, the sensor connector may be a custom connector with the capacity for a plurality of pins. These pins may allow the wire to contain multiple electrical connections to the sensing device. The custom connector may be touch-proof, preventing accidental damage to the device or harm to the subject or carers. The custom connector may also prevent accidental removal of the device when in use.

In another example, the sensor connector may take the form of a TRRS audio jack of 3.5 mm or 2.5 mm. This would enable a firm connection with no risk of accidental removal. The sensor connector of the transmission means may lock into place to prevent the inadvertent removal of the sensor wire.

Alternatively, the transmission means between the device and the sensor device may be wireless. For example, the sensor device may connect to the device by a transmission means such as radio, Bluetooth™ or other methods.

The sensor connector may optionally comprise further circuitry, which may be in the form of a daughter board. This daughter board may comprise a PCB, and be operable to connect the sensor wire to the main PCB. This daughter board would provide modularity to the design. This may feature a 3, 4, 5, or 6 pin design. These connections may allow the sensor device to have multiple electrical connections to the sensor device, including electromagnetic shielding.

In one embodiment, the daughter board may comprise a Kelvin connected resistor to provide improved sensor current acquisition whilst also providing protection from electrical shock from the device to the subject.

If the transmission means is wireless, preferably there is a “back-up” wired system that can be used if the wireless transmission fails or is compromised.

### Connection to External Computing Device

The device may be connected to an external computing device by a communication connection. The communication connection is operable to transfer a processed electroanalytical signal, preferably an amplified and/or digital electroanalytical signal, and a processed electrophysiological signal, preferably an amplified and/or digital electrophysiological signal, from the device to the external computing device.

The communication connection between the device and the external computing device may be wired or wireless.

In one embodiment, the communication connection between the device and the external computing device may be wireless, to allow transmission of the processed electroanalytical signal indicative of the concentration of an analyte and the processed electrophysiological signal indicative of an ECG waveform of the subject. This may take the form of radio waves, Bluetooth™, WIFI, 3G, 4G, 5G or other methods. A person skilled in the art will appreciate that this form of communication may take many different forms.

Alternatively, the communication connection between the device and the external computing device may comprise wires and one or more output connectors, to allow transmission of the processed electroanalytical signal indicative of the concentration of an analyte and the processed electrophysiological signal indicative of an ECG waveform of the subject. The device may have an output connector with a matching output connector on the communication connection wires, such that the communication connection wires are able to be plugged in and may be removed. Alternatively, the communication connection wires may be integral to the electronic processing unit such that the communication connection wires are not removable.

The other end of the communication connection wires (to be connected to the external computing device) may have an output connector and a matching output connector on the external computing device, such that the communication connection wires are plugged in and may be removed. Alternatively, the communication connection wires may be integral to the external computing device such that the communication connection wires are not removable.

It is preferable that at least one end of the communication connection wires comprises an output connector, with a corresponding output connector on the device or external computing device. Optionally both ends of the communication connection wire comprise an output connector, such that the communication connection wires/output connectors may be replaced without replacing the device and/or external computing device.

In one embodiment, the output connector may take the form of a USB B connection but one skilled in the art could appreciate that a multitude of different connections are possible, including but not limited to USB A, USB C, and micro USB. A person skilled in the art could appreciate that any form of connector may be used. Common connector formats may be used to increase compatibility of the electronic processing unit with a range of external computing devices. Alternatively, a distinct or uncommon connector type may be utilized to prevent the use of unsuitable devices as the external computing device. The output connector at each end of the communication connection wire may be different, such that the output connector at the device end of the communication connection wires is of one type and the output connector at the external computing device end of the communication connection wires may be of another type.

In one embodiment, the communication connection wires (and output connection(s)) may further function as a connection between the device and an energy source. If the output connection and wires of the device are connected to an external computing device, the power source may be the external computing device. The external computing device may provide the energy source for the device, or may be used to charge rechargeable batteries within the device.

In one embodiment, the components of the device may be separated, such as being housed in separate boxes, patches etc, such that the PCB/PCBA, energy source, and communication connection components are separate to the accelerometer, sensor wires and strap components. This would enable a smaller ‘dumb’ part of the device to be placed on the body of the subject while a larger ‘smart’ part of the device may be placed elsewhere. In one embodiment, the components of the device may be separated such that the accelerometer, ECG ground electrode (EGE) and strap components are attached to the body of the mother if the subject is a fetus, whilst the PCB/PCBA, energy source and communication connection components are provided in a separate location, connected to the first set of components via a wire or cable.

In one embodiment, the device may also comprise a radio-frequency identification (RFID) tag. This could allow the device to be paired with sensor devices or external computing devices. In one embodiment, the device may also comprise a near-field communication (NFC) tag operable to allow the device to be paired with sensor devices or external computing devices.

Any suitable communication protocol can be used to facilitate connection and communication between any subsystems or components of the device, and other devices or systems, as are well known to persons skilled in the art and need not be described in any further detail herein except as is relevant to the present invention.

### Connection Studs

The device of the present invention may comprise connection studs in the form of studs or protrusions that may function as one or more of the following:
a ground electrode for the ECG (such as a maternal ground electrode);
an anti-movement protrusion during charging and use;
a wired charging point; and/or
a connection for data transfer.

In one embodiment, there may be three studs, but other embodiments can be imagined, such as 1, 2, 3, 4 or more studs. Preferably the studs are made of metal, or other conductive material. In an embodiment, the studs may take the form of pogo pins.

In one embodiment, the studs may provide the ECG ground electrode (EGE) which may connect to the skin of the subject or the mother if the subject is a fetus via an adhesive patch, strap, or bandage. This would connect to the EGE in the PCB/PCBA.

In this embodiment, the connection of the EGE to the mother would enable the filtering of common mode noise introduced by mains electricity in circuits in nearby electronic devices.

In this embodiment, the studs which provide the ECG ground electrode (EGE) may optionally include, or be provided with, a conductive gel to improve conductivity. This conductive gel may be applied either on the studs or on the adhesive patch, strap, or bandage.

In one embodiment, the studs may function as anti-movement protrusions to provide firm purchase for the device on a charging holder, for example on the external computing device, providing a secure place to leave spare devices.

In one embodiment, the studs may act as anti-movement protrusions to hold the device to the adhesive patch, strap, or bandage when in use.

In one embodiment, the studs may allow for wired charging of the device on the charging holder. The wired charging may use the metal (or other conductive material) of the studs to provide a connection for charge to pass through. Alternatively wireless charging may be possible.

In one embodiment, the studs may allow for data transfer between the device and the external computing device.

In one embodiment, each stud may perform one or more of the following actions: data transfer, ECG ground electrode (EGE), charging of the power source, and providing firm purchase on the charging holder. A person skilled in the art would appreciate that this can be achieved through a system of transistors, multiplexers, or physical switches. This would enable each stud to be multifunctional. Alternatively, one or more studs may have a single function, one or more studs may be multifunctional, whilst other studs may be inactive.

### Adhesive Pad, Bandage or Strap

The device may be secured to the body of the subject by an adhesive patch, strap, or bandage. This adhesive patch, strap, or bandage may be secured to the arm or leg of the subject. A person skilled in the art would appreciate that the device may be secured to any suitable part of the body. The electronic processing unit may be secured to the body of the subject by a removable adhesive patch, strap, or bandage. In one embodiment, the device may be secured to the leg or belly of the mother of the fetus if the subject is a fetus by an adhesive patch, strap, or bandage. The electronic processing unit may also be secured to the body of the mother by a removable adhesive patch, strap, or bandage.

In one embodiment, the adhesive patch, strap, or bandage may include pad studs to connect to the connection studs. This would prevent undesirable movement of the device on the mother. This would also allow the ECG ground electrode to touch the mother, allowing for filtering of common mode noise.

### Top Cover

In one embodiment, the device comprises a top cover over the PCB/PCBA and other elements of the device. The top cover will cover and protect the vulnerable areas of the device.

In one embodiment, the top cover may be composed of plastic, either flexible or rigid. The materials used may include various plastics such as silicone, polycarbonate, polyurethane, polypropylene, polyethylene, polyethylene terephthalate, poly (methyl methacrylate) (PMMA), metals or others. One skilled in the art would appreciate that the top cover may be composed of any suitable plastic or other material.

In one embodiment, the top cover may contain a material designed to stop the transmission of electromagnetic fields. This is commonly referred to as a Faraday cage. This would reduce the risk of interference by electromagnetic fields with the device. This material must be conductive and may comprise a mesh or solid film. Any suitable conductive material may be used, such as steel or copper. It may be flexible.

### Bottom Cover

In one embodiment, the device comprises a bottom cover over the PCB/PCBA and other elements of the device. The bottom cover will cover and protect the vulnerable areas of the device. The materials used may include various plastics such as silicone, polycarbonate, polyurethane, polypropylene, polyethylene, polyethylene terephthalate, poly (methyl methacrylate) (PMMA), metals or others. One skilled in the art would appreciate that the bottom cover may be composed of any suitable plastic or other material.

In one embodiment, the bottom cover may contain a material designed to stop the transmission of electromagnetic fields. This is commonly referred to as a Faraday cage. This would reduce the risk of interference by electromagnetic fields with the device. This material must be conductive and may comprise a mesh or solid film. Any suitable conductive material may be used, such as steel or copper among many others. It may be flexible.

### External Computing Device

The device may be in communication with an external computing device.

The external computing device may comprise a display that allows for the simultaneous, or near simultaneous, display of relevant data and/or information, such as trend of baseline concentration of an analyte and/or the electrocardiogram (ECG), for example. The external computing device may optionally be configured to display a visual and/or or auditory warning if the concentration of the analyte and/or the ECG drop beneath a certain threshold. The external computing device may optionally be configured to display the absolute concentration of an analyte in the subject.

The external computing device may include a charging stand for the device when not in use. This stand may allow for wireless or wired charging of the device.

The external computing device may take the form of a mobile computing device. The mobile computing device may be a communication device and may comprise a smartphone such as that marketed under the trademark IPHONE® by Apple Inc, or by other provider such as Nokia Corporation, or Samsung Group, having Android, WEBOS, Windows, or other Phone app platform. Alternatively, the external computing device may comprise other computing means such as a personal, notebook or tablet computer such as that marketed under the trademark IPAD® or IPOD TOUCH® by Apple Inc, or by other provider such as Hewlett-Packard Company, or Dell, Inc, for example, or other suitable device.

### System for Processing

The invention further provides a system for processing an electroanalytical signal indicative of concentration of an analyte in a subject and an electrophysiological signal of the subject, the system comprising:
a) a sensor device attachable to the subject, said sensor device comprising (i) a biosensor device capable of and operable for sensing the concentration of an the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of the subject;
b) a transmission means to transmit the electroanalytical signal and the electrophysiological signal to a device for processing signals indicative of health; and
c) any embodiment of a device for processing signals indicative of health according to the first broad aspect of the present invention, or as herein described.

The invention further provides a system for processing an electroanalytical signal indicative of concentration of an analyte in a fetus and an electrophysiological signal of the fetus, the system comprising:
a) a fetal sensor device attachable to the fetus, said fetal sensor device comprising (i) a biosensor device capable of and operable for sensing the concentration of an the analyte in the fetus and (ii) an electrocardiogram (ECG) capable of and operable for sensing an ECG waveform of the fetus, wherein said biosensor device of the fetal sensor device generates an electroanalytical signal indicative of the concentration of the analyte in the fetus and said ECG of the fetal sensor device generates an electrophysiological signal of the fetus;
b) a transmission means to transmit the electroanalytical signal and the electrophysiological signal to a device for processing signals indicative of health; and
c) any embodiment of a device for processing signals indicative of health according to the first broad aspect of the present invention, or as herein described.

### Method for Processing

The invention further provides a method for processing an electroanalytical signal indicative of the concentration of an analyte in a subject and an electrophysiological signal of the subject, the method comprising the steps of:
a) attaching a sensor device to the subject, said sensor device comprising (i) a biosensor device capable of and operable for sensing the concentration of the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of the subject;
b) transmitting the electroanalytical signal and the electrophysiological signal to a device for processing signals indicative of health via a transmission means; and processing the electroanalytical signal and the electrophysiological signal in the device for processing signals indicative of health, wherein the device for processing signals indicative of health comprises any embodiment of a device for processing signals indicative of health according to the first broad aspect of the present invention, or as herein described.

The invention further provides a method for processing an electroanalytical signal indicative of concentration of an analyte in a fetus and an electrophysiological signal of the fetus, the method comprising the steps of:
a) attaching a fetal sensor device to the fetus, said fetal sensor device comprising (i) a biosensor device capable of and operable for sensing the concentration of the analyte in the fetus and (ii) an electrocardiogram (ECG) capable of and operable for sensing an ECG waveform of the fetus, wherein said biosensor device of the fetal sensor device generates an electroanalytical signal indicative of the concentration of the analyte in the fetus and said ECG of the fetal sensor device generates an electrophysiological signal of the fetus;
b) transmitting the electroanalytical signal and the electrophysiological electronic signal to a device for processing signals indicative of health via a transmission means; and
c) processing the electroanalytical signal and the electrophysiological signal in the device for processing signals indicative of health, wherein the device for processing signals indicative of health comprises any embodiment of a device for processing signals indicative of health according to the first broad aspect of the present invention, or as herein described.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement, and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The term “real-time”, for example “displaying real-time data,” refers to the display of the data without intentional delay, given the processing limitations of the system and the time required to accurately measure the data.

The term “near-real-time”, for example “obtaining real-time or near-real-time data” refers to the obtaining of data either without intentional delay (“real-time”) or as close to real-time as practically possible (i.e. with a small, but minimal, amount of delay whether intentional or not within the constraints and processing limitations of the of the system for obtaining and recording or transmitting the data. The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the invention are to be provided in the following non-limiting Examples. The description is included solely for the purposes of exemplifying the present invention. It should not be understood as a restriction on the broad invention set out above.

In the Examples, the following abbreviations will be used:
Biosensor working electrode (BWE)
Biosensor reference electrode (BRE)
Biosensor counter electrode (BCE)
ECG working electrode (EWE)
ECG reference electrode (ERE)
ECG ground electrode (EGE)
Printed circuit board (PCB). This term is taken to also include a printed circuit board assembly (PCBA), referring to a complete and functional printed circuit board containing all of the necessary electronic components operably in place and connected so as to function as described.

### Example 1

### Device

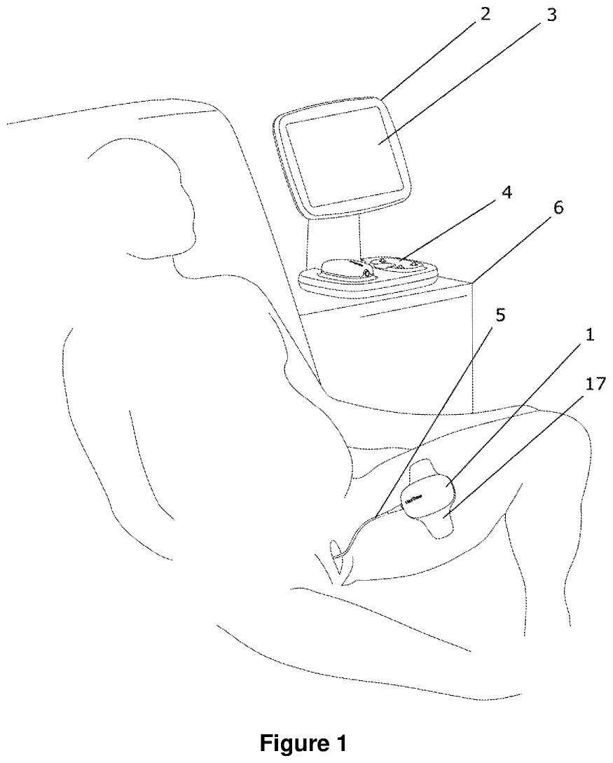

Referring now to the drawings, FIG. 1 shows a device for processing signals indicative of health according to an embodiment of the invention in the form of an electronic processing unit 1 connected via sensor wire 5 to a sensor device attached to the fetus in the womb. A person skilled in the art will appreciate that electronic processing unit 1 may be placed on any suitable part of the mother's body. Electronic processing unit 1 is in wireless connection to external computing device 2. External computing device 2 may comprise a charger in the form of a charging holder 4. In this Figure, external computing device 2 is shown charging additional electronic processing units equivalent to electronic processing unit 1 on charging holder 4. FIG. 1 also shows adhesive pad 17, which allows electronic processing unit 1 to be secured to the mother's body. A person skilled in the art will appreciate that electronic processing unit 1 may be placed on any suitable part of the body.

External computing device 2 may be placed on a bedside 6 or other suitable surface, as illustrated, to provide a clinician with immediate access to the data provided, but one skilled in art would appreciate that external computing device 2 may be placed anywhere within range of electronic processing unit 1.

In one embodiment, the adhesive pad 17 may be replaced by a removable strap which holds the electronic processing unit 1 to the mother's body. A person skilled in the art would appreciate that this may be placed on any suitable part of the mother's body.

In one embodiment, external computing device 2 may comprise a display 3 for displaying relevant data and/or information, such as data and/or information indicative of the concentration of the analyte and the ECG waveform of the fetus. The information on display 3 may be of any suitable form. For example, the absolute concentration of the analyte may be displayed using numbers or a graph. A heart rate may be provided as a graph of voltage versus time of the electrical activity of the heart. In this way, a relevant medical professional, such as a doctor or midwife, can monitor, via the display 3, the concentration of the analyte and the ECG waveform continuously and in real-time. Thus, if the information indicative of the concentration of an analyte exceeds or falls below a predetermined threshold or the heart rate changes, the doctor or midwife can intervene immediately.

The device, implemented in the electronic processing unit 1, comprises a plurality of components, subsystems and/or modules operably coupled via appropriate circuitry and connections to enable the device to perform the functions and operations herein described. In the present embodiments this is implemented by a main printed circuit board (assembly) (PCB) 8. In alternative embodiments, this may be implemented by other suitable technology, such as wire wrap or point to point construction.

As will be described in further detail, the circuitry of the device is operable to: receive input comprising a first signal indicative of health and a second signal indicative of health; process the first signal indicative of health; process the second signal indicative of health; and provide an output arising from the processing of the first signal indicative of health and the second signal indicative of health.

In the embodiments, the first signal indicative of health comprises an electroanalytical signal indicative of concentration of an analyte in a fetus.

In the embodiments, the second signal indicative of health comprises an electrophysiological signal of the fetus.

In the embodiments, the circuitry comprises at least two circuits, the first circuit being an electroanalytical signal processing circuit adapted to process the electroanalytical signal indicative of the concentration of the analyte in the fetus, and the second circuit being an electrophysiological signal processing circuit adapted to process the electrophysiological signal of the fetus.

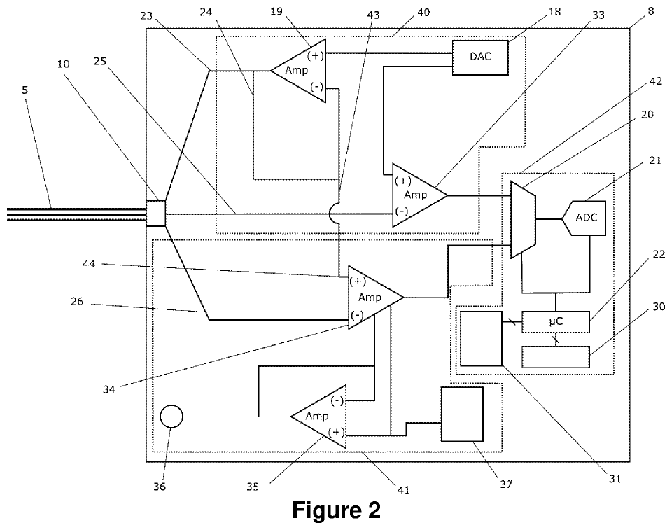

Referring now to FIGS. 2 to 7, FIGS. 2 to 7 show first to sixth arrangements of the main PCB 8 in accordance with the embodiments of the present invention.

The main PCB 8 shown in FIGS. 2 to 7 comprises sensor connector 10 for receiving, or the inputting of, signals, from the fetal sensor device, carrying signal representative of, for example, vital signs of the fetus and mother as well as maternal movements (as well as fetus movement indirectly), a plurality of modules 40, 41 and 42 for processing the signals received from the fetal sensor device, an output means 31 (part of module 42) for delivering, or outputting, the processed signals (which may comprise data and/or information) to the external computing device 2. Each of modules 40, 41 and 42 includes a respective circuit for processing of at least one signal. In particular, the circuits of modules 40 and 41 process the signal from the fetal sensor device and the circuit of module 42 processes the signals exiting modules 40 and 41.

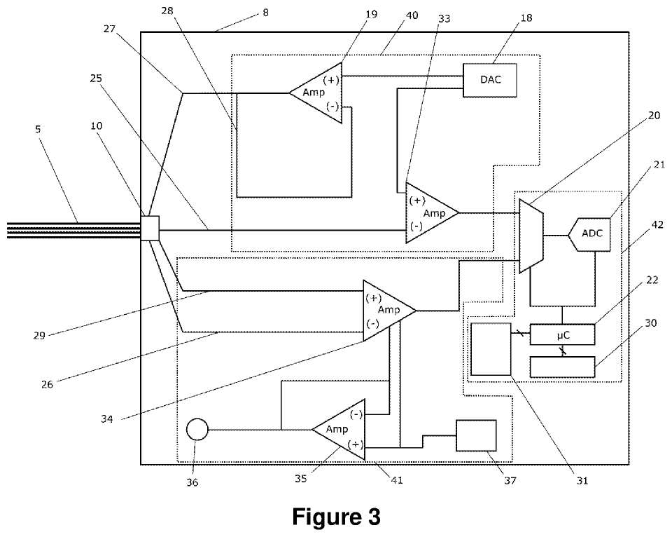

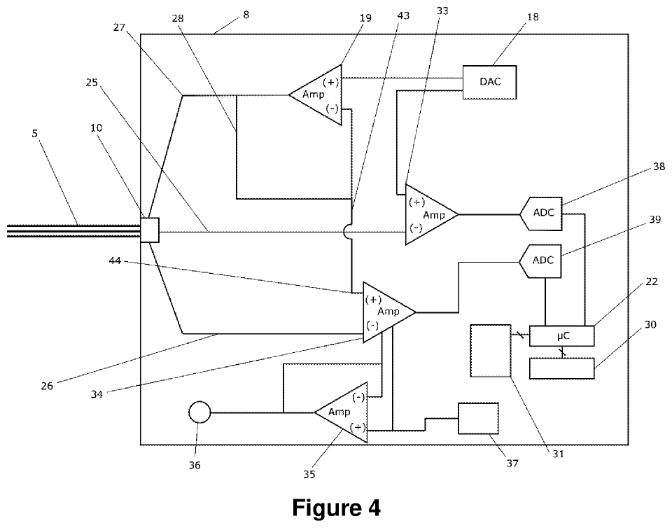

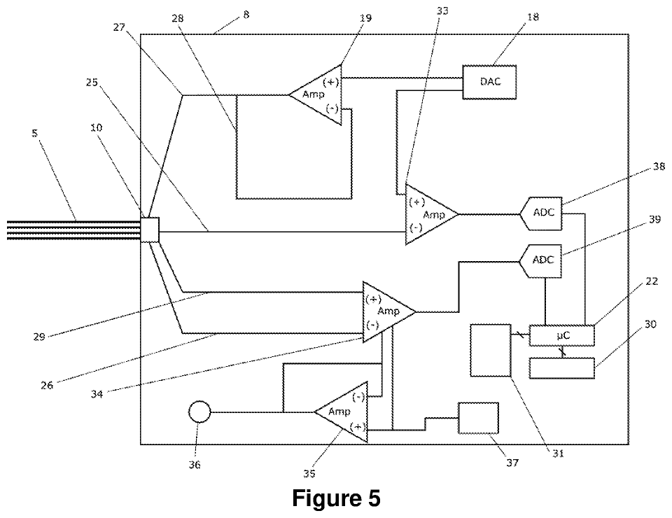

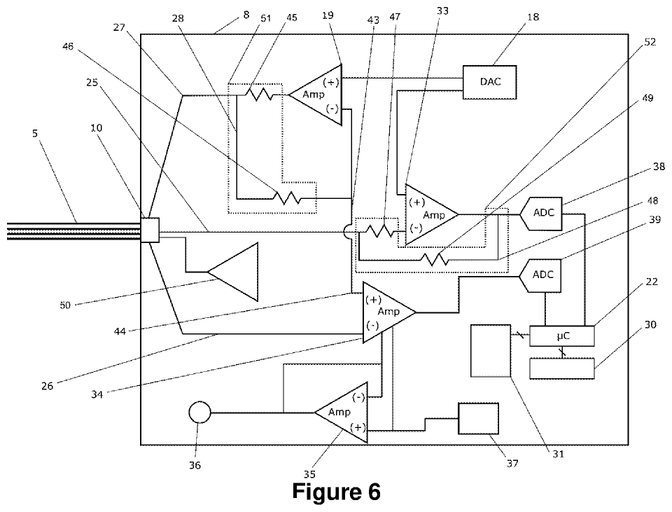

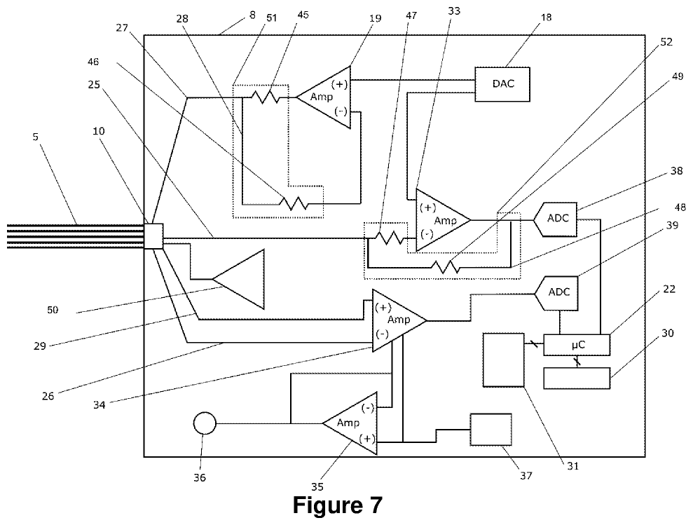

The sensor connector 10 may be a connector for receiving a wire 5 electrically connected to the fetal sensor device. As shown in FIGS. 2 and 4, the wire 5 comprises a 3-core cable comprising cables electrically connected to the BWE, the EWE/BRE and the ERE. As shown in FIGS. 3 and 5, the wire 5 comprises a 4-core cable comprising cables electrically connected to the BWE, the EWE, the BRE and the ERE. As shown in FIG. 6, the wire 5 comprises a 4-core cable comprising cables electrically connected to the BWE, the EWE/BRE, the ERE, and the shielding amplifier 50. As shown in FIG. 7, the wire 5 comprises a 5-core cable comprising cables electrically connected to the BWE, the EWE, the BRE, the ERE, and the shielding amplifier 50.

In alternative arrangements, the sensor connector 10 may be by wireless transmission means such as radio, Bluetooth™, or other methods referred to above.

In an alternative arrangement, sensor connector 10 may comprise a daughter board (not shown). This daughter board would be a PCB, capable of connecting the wire 5 to the main PCB 8.

The modules 40 and 41 are adapted to process the signal (which may comprise data and/or information), respectively, from the biosensor device and the ECG of the fetal sensor device. Module 42 is adapted to receive and process signals from modules 40 and 41.

As discussed above, there are six arrangements of main PCB 8. The first and second arrangements of main PCB 8 differ from the third and fourth arrangements of main PCB 8 in that the first and second arrangements comprise a multiplexer 20 for generating a single output that is fed to a single analog-to-digital convertor (ADC) 21, and the third and fourth arrangements omit use of a multiplexer due to having an ADC (ADC 38 and ADC 39) for each of the signals exiting the modules 40 and 41. The fifth and sixth arrangements differ from the third and fourth arrangements by possessing two sets of Kelvin connected resistors 51 and 52 and a shielding amplifier 50. The BRE Kelvin connected resistor 51 is formed of BRE/BCE resistor 45 and BRE resistor 46 and the BWE Kelvin connected resistor 52 is formed from BWE resistor 47, BWE amplifier wire 48, and BWE amplifier resistor 49.

The first, third, and fifth arrangements of main PCB 8 differ with respect to the second, fourth, and sixth arrangement of main PCB 8 in that the first, third, and fifth arrangements use a single reference voltage for operation of both modules 40 and 41. Wire 43 makes this connection between module 40 and module 41 and connects the negative terminal of amplifier 19 and the positive terminal of amplifier 34. This allows for the sharing of the reference voltage between modules 40 and 41. This allows for only three or four wires in wire 5, which advantageously enables the fetal sensor device to use fewer electrodes and be less invasive than would otherwise be required.

In contrast, the second, fourth, and sixth arrangements of main PCB 8 use two separate reference voltages provided by the BRE and the ERE of the fetal sensor device to operate modules 40 and 41 respectively. In particular, the BRE/BCE reference voltage is fed to module 40 and the ERE reference voltage is fed to module 41. In the second, fourth, and sixth arrangements of main PCB 8, the wire 5 comprises at least four cables: a cable carrying the BRE/BCE signal; a cable carrying the BWE signal; a cable carrying the ERE signal; and a cable carrying the EWE signal. In FIGS. 3, 5, and 7 via the connector 10, the cable carrying the BRE signal is electrically connected to cable 27, the cable carrying the BWE signal is electrically connected to cable 25, the cable carrying the EWE signal is electrically connected to cable 26, and the cable carrying the ERE signal is electrically connected to cable 29.

FIG. 2 shows the first arrangement of the layout of main PCB 8. FIG. 2 shows three modules: the biosensor module 40, the ECG module 41, and the microcontroller module 42.

The biosensor module 40 is formed of BRE/BCE/EWE wire 23, which connects to sensor connector 10 through to sensor cable 5. This forms one of the three wires within cable 5. The biosensor module 40 is also comprised of BWE wire 25, which connects to sensor connector 10 through to sensor cable 5. This forms another of the three wires within cable 5. BRE/BCE/EWE wire 23 splits to connect to biosensor amplifier 19 and wire 24. Wire 24 connects to the negative terminal of biosensor amplifier 19. This creates a digitally controlled bias voltage between the BRE/BCE/EWE wire 23 and BWE wire 25, thus transducing a current signal at amplifier 33. Wire 24 further splits to form wire 43, which connects to the non-inverting positive pin 44 of ECG amplifier 34. Wire 43 carries a common reference voltage created by biosensor module 40 to ECG module 41, allowing both modules to share a common reference voltage. This reduces the number of wires required for a connection to the sensor device from four to three. The positive terminal biosensor amplifier 19 is connected to digital-to-analog converter (DAC) 18. DAC 18 further connects to the positive terminal of biosensor amplifier 33. The negative terminal of biosensor amplifier 33 connects to BWE wire 25, while the output of the amplifier connects to multiplexer 20, connecting biosensor module 40 to microcontroller module 42.

The ECG module 41 is formed of EWE wire 26, which connects sensor connector 10 through to sensor cable 5. This forms the final wire of the three wires within cable 5. EWE wire 26 further connects to the negative terminal of ECG amplifier 34. The positive terminal of ECG amplifier 34 is connected to wire 43, which provides the common reference voltage shared by module 40. The output of ECG amplifier 34 connects to microcontroller module 42 via multiplexer 20. The EGE 36 is attachable to and connects to the body of the patient (i.e. the mother, not shown) and provides a means for removing common mode noise. The EGE 36 connects to EGE amplifier 35, whose positive terminal connects to virtual ground 37. The negative terminal of EGE amplifier 35 connects to ECG amplifier 34. The ECG module 41 thus formed can measure ECG signals between wire 43 and wire 26, whilst filtering common mode noise using EGE 36.

The microcontroller module 42 is formed by multiplexer 20, which connects to analog-to-digital converter (ADC) 21. Multiplexer 20 is connected to biosensor module 40 and ECG module 41 via amplifiers 33 and 34 respectively. This arrangement allows for rapid switching between both modules providing near simultaneous processing of the electroanalytical signal indicative of the concentration of an analyte of the fetus and the ECG wave form generating an electrophysiological signal of the fetus. ADC 21 is further connected to microcontroller 22, which connects to accelerometer 30 and wireless communication device 31.

In one embodiment, the amplifiers 19, 33, 34, and 35 may all have filters (not shown) to remove undesirable frequencies to improve the electronic signal received.

The sensor connector 10 is shown connected to sensor cable 5, which comprises the wires carrying the BWE, BRE/BCE, EWE, and ERE signals. Shown is microprocessor 22, which delivers electronic signals to wireless communication device 31 and multiplexer 20. The device receives signals from accelerometer 30 and the analog-to-digital converter 21. Multiplexer 20 is connected to biosensor amplifier 33 and ECG amplifier 34. This arrangement allows for near simultaneous analysis of both signals. ECG amplifier 34 is also connected to ECG (EGE) amplifier 35, which further connects to ECG (EGE) 36. The ECG ground electrode (EGE) amplifier 35 further connects to virtual ground 37. The negative terminal of biosensor amplifier 33 takes the signal from the fetal sensor device via sensor wire 5 and through sensor connector 10 via biosensor working electrode (BWE) wire 25. The positive terminal of biosensor amplifier 33 is connected to digital-to-analog converter 18 which connects to biosensor reference amplifier 19. This creates a digitally controlled bias voltage between the BRE/BCE wire 23 and BWE wire 25, thus transducing a current signal at amplifier 33. The negative terminal of biosensor reference amplifier 19 is connected to biosensor reference electrode (BRE) wire 24. This connection branches to form wire 43. Wire 43 carries a common reference voltage provided by module 40 to the positive terminal of 34. This allows the ECG circuit 41 to use the same reference voltage as biosensor module 40.

The electrical reference signal from the fetal sensor device (not shown) passes through sensor cable 5 via the sensor connector 10 via wire 23 to wire 24 and amplifier 19. The negative terminal of ECG amplifier 34 is connected to the fetal sensor device via wire 26—picking up the ECG signal. The positive terminal of the ECG amplifier 34 is connected to the combined outputs of the negative terminal of biosensor amplifier 19 and the BRE wire 24 through the wire 43.

In this embodiment, the ECG circuit of ERE wire 26, amplifier 34, amplifier 19, BRE wire 24, BCE/BRE/EWE wire 23, EGE amplifier 35, EGE 36, and virtual ground 37 has a common reference voltage provided by wire 43. The biosensor circuit of amplifier 33, digital-to-analog converter (DAC) 18, amplifier 19, BCE/BRE/EWE wire 23, BRE wire 24, and BWE wire 25 shares that same reference voltage. This reduces the number of wires required for a connection to the sensor device from four to three.

In one embodiment, the wires connecting to amplifiers 19, 33, 34, and 35 may all include filters (not shown) to remove undesirable frequencies and improve the signal received.

FIG. 3 shows an alternative second arrangement of main PCB 8 with three modules: the biosensor module 40, the ECG module 41, and the microcontroller module 42.

The biosensor module 40 is formed of BRE/BCE wire 27, which connects to sensor connector 10 through to sensor cable 5. This forms one of the four wires within cable 5. The biosensor module 40 is also comprised of BWE wire 25, which connects to sensor connector 10 through to sensor cable 5. This forms another of the four wires within cable 5. BRE/BCE wire 27 splits to connect to biosensor amplifier 19 and wire 28. Wire 28 connects to the negative terminal of biosensor amplifier 19. This creates a digitally controlled bias voltage between the BRE/BCE wire 27 and BWE wire 25, thus transducing a current signal at amplifier 33. This arrangement provides a reference voltage only accessible by biosensor module 40, requiring a separate reference voltage for ECG module 41. The positive terminal biosensor amplifier 19 is connected to digital-to-analog converter (DAC) 18. DAC 18 further connects to the positive terminal of biosensor amplifier 33. The negative terminal of biosensor amplifier 33 connects to BWE wire 25, while the output of the amplifier connects to multiplexer 20, connecting biosensor module 40 to microcontroller module 42.

The ECG module 41 is formed of EWE wire 26, which connects to sensor connector 10 through to sensor cable 5. This forms the third wire of the four wires within cable 5. EWE wire 26 further connects to the negative terminal of ECG amplifier 34. The positive terminal of ECG amplifier 34 is connected to EWE wire 29, which provides a separate reference voltage for ECG module 41. The output of ECG amplifier 34 connects to microcontroller module 42 to multiplexer 20. The EGE 36 connects to the body of the patient (i.e. the mother, not shown) and provides a means for removing common mode noise. The EGE 36 connects to EGE amplifier 35, whose positive terminal connects to virtual ground 37. The negative terminal of EGE amplifier 35 connects to ECG amplifier 34. The ECG module 41 thus formed can measure ECG signals between EWE wire 29 and ERE wire 26, whilst filtering common mode noise using EGE 36.

The microcontroller module 42 is formed by multiplexer 20, which connects to analog-to-digital converter (ADC) 21. Multiplexer 20 is connected to biosensor module 40 and ECG module 41 via amplifiers 33 and 34 respectively. This arrangement allows for rapid switching between both modules providing near simultaneous processing of the electroanalytical signal indicative of the concentration of an analyte of the fetus and the ECG wave form generating an electrophysiological signal of the fetus. ADC 21 is further connected to microcontroller 22, which connects to accelerometer 30 and wireless communication device 31.

In one embodiment, the amplifiers 19, 33, 34, and 35 may all have filters (not shown) to remove undesirable frequencies to improve the electronic signal received.

In an alternative embodiment, FIG. 3 shows the layout of main PCB 8 wherein the sensor connector 10 is connected to sensor wire 5, which comprises cables carrying BWE, BRE, EWE and ERE signals. It shows microprocessor 22, which delivers to wireless communication device 31 and receives signals from accelerometer 30 and the multiplexer 20 and the analog-to-digital (ADC) converter 21. Multiplexer 20 is connected to biosensor amplifier 33, to which, via the positive terminal, is connected to the digital-to-analog converter (DAC) 18. DAC converter 18 is connected to the positive terminal of biosensor reference amplifier 19. The negative terminal of biosensor reference amplifier 19 is connected to BRE wire 28 which joins to the combined biosensor counter/reference electrode wire (BCE/BRE) 27. The BCE/BRE wire 27 also connects to amplifier 19. The BCE/BRE wire 27 connects to the fetal sensor device via sensor connector 10 and sensor wire 5.

Biosensor amplifier 33 is connected, via the negative terminal, to the fetal sensor device (not shown) by biosensor working electrode (BWE) wire 25, sensor connector 10, and sensor wire 5. Multiplexer 20 is also connected to ECG amplifier 34. The positive terminal of ECG amplifier 34 is connected to ECG working electrode (EWE) wire 29, which connects to the fetal sensor device via sensor connector 10 and sensor wire 5. The negative terminal of amplifier 34 is connected to ECG reference electrode (ERE) wire 26, which connects to the sensor device via sensor connector 10 and sensor wire 5. ECG amplifier 34 is also connected to ECG ground electrode (EGE) amplifier 35, which further connects to ECG ground electrode (EGE) 36. The ECG ground electrode (EGE) amplifier 35 further connects to virtual ground 37.

In this embodiment of the design, the ECG circuit formed of EWE wire 29, ERE wire 26, amplifier 34, EGE amplifier 35, EGE 36, and virtual ground 37 is separate from the biosensor circuit formed of BWE wire 25, BCE/BRE wire 27, BRE wire 28, amplifier 19, digital-to-analog converter 18 and amplifier 33. This provides better separation between the two systems but requires more wires to be run to the fetal sensor device.

In one embodiment, the wires connecting to amplifiers 19, 33, 34, and 35 may all include filters (not shown) to remove undesirable frequencies and improve the signal received from the fetal sensor device.

In the alternative third embodiment shown in FIG. 4, the layout of main PCB 8 is similar to that of FIG. 2 but excludes multiplexer 20 and includes two separate analog-to-digital converters (ADC) 38 and 39. This provides truly simultaneous processing of the electroanalytical signal indicative of the concentration of an analyte of the fetus and the ECG wave form generating an electrophysiological signal of the fetus.

In the alternative fourth embodiment shown in FIG. 5, wherein the layout of main PCB 8 is similar to that of FIG. 3 but excludes multiplexer 20 and includes two separate analog-to-digital converters (ADC) 38 and 39. This provides truly simultaneous electroanalytical signal indicative of the concentration of an analyte of the fetus and the ECG wave form generating an electrophysiological signal of the fetus.

FIG. 6 shows an alternative fifth embodiment, with a main PCB 8 layout similar to FIG. 4. This embodiment provides improved signal acquisition as compared to FIG. 4 by providing two sets of Kelvin connected resistors and a shielding amplifier for sensor wire 5. The BRE Kelvin connected resistor 51 is formed by BRE/BCE resistor 45 and BRE resistor 46. The BWE Kelvin connected resistor 52 is formed by BWE resistor 47, BWE amplifier wire 48, and BWE amplifier resistor 49. By placing the BCE/BRE resistor 45 after the connection of wire 27 and wire 28, this prevents or mitigates electrical shock of the subject, while also improving signal acquisition from the sensing device. The circuit formed by BWE resistor 47, BWE amplifier wire 48, and BWE amplifier resistor 49 provides a similar benefit, preventing or mitigating electrical shock of the subject. The shielding amplifier 50 provides additional shielding from electrical capacitance in the sensor wire 5, by providing a further cable within the sensor wire 5. One skilled in the art would understand that the shielding amplifier 50 may take many forms. The shielding amplifier may prevent the interference of electromagnetic radiation on the signals received by the device.

FIG. 7 shows an alternative sixth embodiment, wherein the layout of main PCB 8 is similar to that of FIG. 5. This embodiment provides improved signal acquisition as compared to FIG. 5 by providing two sets of Kelvin connected resistors and a shielding amplifier for sensor wire 5. The BRE Kelvin connected resistor 51 is formed by BRE/BCE resistor 45 and BRE resistor 46. The BWE Kelvin connected resistor is formed by BWE resistor 47, BWE amplifier wire 48, and BWE amplifier resistor 49. By placing the BCE/BRE resistor 45 after the connection of wire 27 and wire 28, this prevents or mitigates electrical shock of the subject, while also improving signal acquisition from the sensing device. The circuit formed by BWE resistor 47, BWE amplifier wire 48, and BWE amplifier resistor 49 provides a similar benefit, preventing or mitigating electrical of the subject. The shielding amplifier 50 provides additional shielding from electrical capacitance in the sensor wire 5, by providing a further cable within the sensor wire 5. One skilled in the art would understand that the shielding amplifier 50 may take many forms. The shielding amplifier may prevent the interference of electromagnetic radiation on the signals received by the device.

Someone skilled in the art can appreciate that the amplifier(s) in FIGS. 2-7 are a composition of filters, gain stages, buffers and input stages.

In FIGS. 2-7, a series of protection diodes (not shown) may be installed between the ECG amplifier 34 and the EGE amplifier 35. This would prevent or mitigate electrostatic discharge events at the connector from damaging the device, improving the safety and performance of the device.

In FIGS. 2,4, and 6, a series of resistors (not shown) may be installed in the cable 26 and between the EGE amplifier 35 and the ECG ground electrode 36. This provides protection to the subject in the event of an electrical failure, preventing or mitigating electrical shock.

In FIGS. 3, 5, and 7, a series of resistors (not shown) may be installed in the cable 26, between the EGE amplifier 35 and the ECG ground electrode 36, and in cable 29. This provides protection to the subject in the event of an electrical failure, preventing or mitigating electrical shock.

In FIGS. 2-7, a series of protection resistors (not shown) may be installed on any connection to the subject to provide protection against electrical failure, preventing or mitigating electrical shock. One skilled in the art would appreciate that the resistors may be placed anywhere in series with the electroanalytical and electrophysiological circuits to provide this protection to the subject.

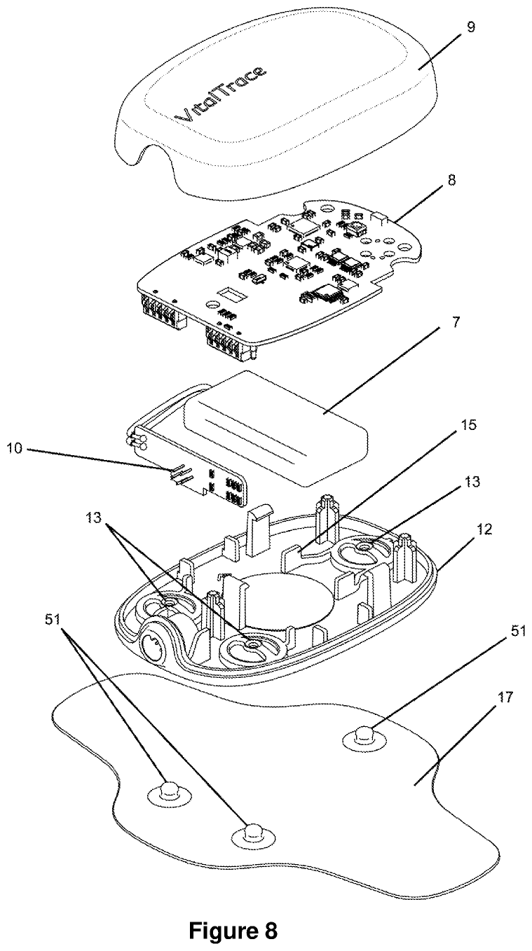

FIG. 8 shows an expanded view of electronic processing unit 1, showing top cover 9, which encloses electronic processing unit 1. Additionally, an energy source in the form of a battery 7 of electronic processing unit 1 is shown. The main printed circuit board (PCB) 8 of electronic processing unit 1 is shown. FIG. 8 also shows sensor connector 10, which allows for wired connection of sensor wire 5 connecting to the fetal sensor device. The internal housing 15 is shown, which provides a holder for the battery 7, main PCB 8, and sensor connector 10. Bottom cover 12 is shown, which combined with top cover 9 provides an enclosure for the device that is electronic processing unit 1. Adhesive pad 17 is shown, allowing for the connection of additional electrodes to the mother's body via the pad studs 51. Studs 13 allow for mounting of the electronic processing unit 1 on charging holder 4 (not shown) and adhesive pad 17.

In one embodiment, the top cover 9 combined with bottom cover 12 may be designed to be substantially waterproof to prevent the ingress of water and other fluids into electronic processing unit 1. This would allow for the device to be used in water births and in wet environments.

In FIG. 8, the energy source in the form of battery 7 is shown as one battery but one skilled in the art could appreciate that the energy source could be two or more batteries, potentially in combination with other energy sources.

In one embodiment, sensor connector 10 may be designed to prevent the ingress of water and other fluids into the electronic processing unit 1. This would allow for the device to be used in water births and in wet environments.

In one embodiment, the internal housing 15, top cover 9, bottom cover 12, and adhesive pad 17 may be designed to be flexible, allowing the electronic processing unit 1 to be flexed over the body of the mother when in use.

In one embodiment, studs 13 may provide a means to hold the electronic processing unit 1 to the charging holder 4 (not shown) to allow for charging either via an electrical connection or by wireless charging of an energy source (i.e. battery 7 in the embodiments).

In one embodiment, the studs 13 would connect to pad studs 51 to provide a connection for the ECG ground electrode 36 (not shown) to touch the skin of the mother's body. This would allow the ECG ground electrode 36 (not shown) to make an electrical connection reliably and easily to the mother's body.

In one embodiment, the studs 13 would allow for data transfer between the electronic processing unit 1 and the external computing device 2 (not shown) via receiver studs 16 (not shown).

In a one embodiment, a selection of studs 13 may each serve individual purposes: as a means for data transfer; as a means for charging the device; and/or as a connection to EGE 36 (not shown) via receiver studs 16 (not shown). This would enable all studs to serve a different purpose.

In this embodiment, the studs 13 and receiver studs 16 (not shown) may be arranged in a pattern that allows for the device to be placed on charging holder 4 (not shown) in one manner only, allowing each of studs 13 to be connected to their respective receiver stud 16 (not shown).

In one embodiment, one stud 13 or more may each have multiple purposes: as a means for data transfer; as a means for charging the device; and as a connection to EGE 36 (not shown) via receiver studs 16 (not shown). Each stud may serve more than one purpose, or may simply act as a way to secure the device to charging holder 4 (not shown). A person skilled in the art could appreciate that these studs could be connected in a multitude of ways.

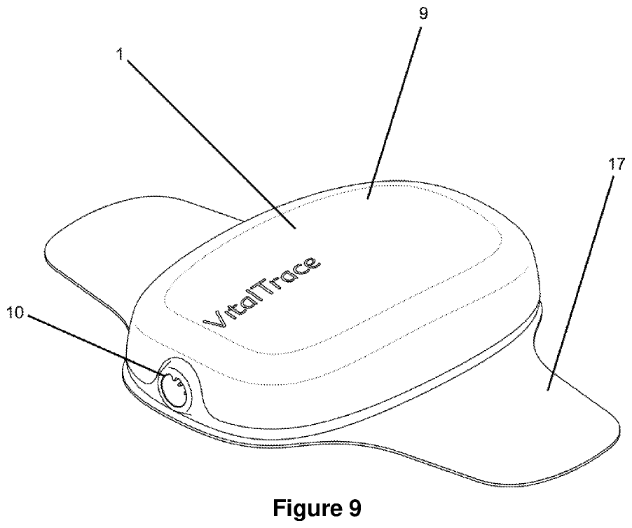

FIG. 9 shows an assembled electronic processing unit 1, complete with top cover 9, and adhesive pad 17.

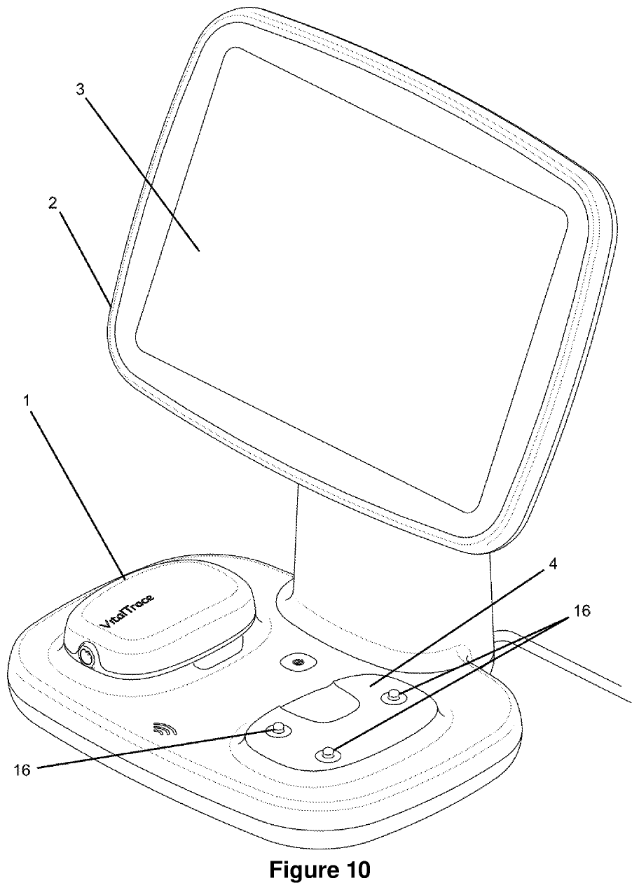

FIG. 10 shows external computing device 2, along with one electronic processing unit 1 shown on charging station 4. Also shown is display 3 and receiver studs 16. The charging station 4 provides a convenient location to place electronic processing unit 1 when not in use.

### Example 2

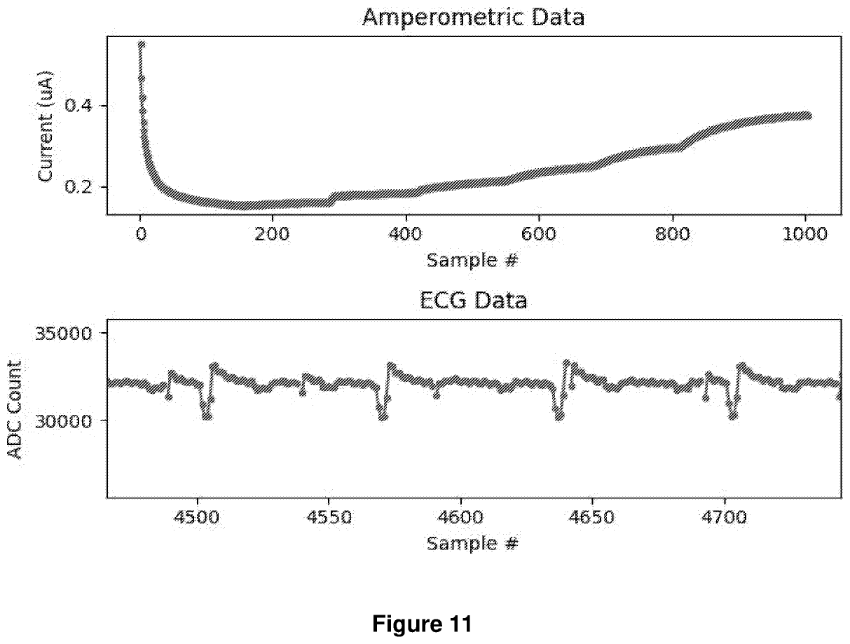

FIG. 11 shows an example of the data obtained from the combination of the device for processing signals indicative of health of the present invention, when used in conjunction with a fetal sensor device providing an electroanalytical signal indicative of the concentration of a lactate analyte in a fetus and an electrophysiological signal of the fetus.

This example shows that an electronic processing unit 1 (such as that described above and provided in FIG. 2) can measure changes in an electroanalytical signal indicative of increases in analyte concentration whilst also measuring an electrophysiological signal indicative of the ECG waveform of the fetus.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A device for processing signals indicative of health in a subject, the device comprising circuitry operable to:
receive input comprising a first signal indicative of health and a second signal indicative of health;
process the first signal indicative of health;
process the second signal indicative of health; and
provide an output arising from the processing of the first signal indicative of health and the second signal indicative of health.

2. The device of claim 1, wherein the first signal indicative of health comprises an electroanalytical signal indicative of concentration of an analyte.

3. The device of claim 1, wherein the second signal indicative of health comprises an electrophysiological signal.

4. The device of any one of the preceding claims, wherein the circuitry comprises at least two circuits, the first circuit being an electroanalytical signal processing circuit adapted to process the electroanalytical signal indicative of the concentration of the analyte in the subject, and the second circuit being an electrophysiological signal processing circuit adapted to process the electrophysiological signal of the subject.

5. The device of any one of the preceding claims, wherein the processing comprises at least one of: amplification; conversion from analogue to digital.

6. The device of claim 4 where in the electroanalytical and electrophysiological processing circuits comprise a resistor arrangement that provides protection from electrical shock.

7. The device of any one of the preceding claims, wherein the device is implemented in a unit, to thereby provide an electronic processing unit for processing an electroanalytical signal indicative of the concentration of an analyte in a fetus and an electrophysiological signal of the fetus.

8. The device of claim 4, wherein the electroanalytical signal processing circuit and the electrophysiological signal processing circuit share a common reference voltage.

9. The device of claim 7, wherein the common reference voltage between the electrophysiological signal processing circuit and the electroanalytical signal processing circuit is in the form of a connection, connecting a first biosensor amplifier component to a second electrophysiology amplifier component which also shares an electrical connection with a biosensor reference electrode and an electrocardiogram electrode.

10. The device of any one of the preceding claims, wherein the circuitry is provided on or otherwise implemented in, a printed circuit board (PCB)/printed circuit board assembly (PCBA).

11. The device of any one of the preceding claims, further comprising one or more of the following:
i. an energy source;
ii. a wired transmission means to transmit an electrophysiological signal and/or an electroanalytical signal from a sensor device to the device; and
iii. a communication connection for the transfer of a processed electrophysiological signal and/or a processed electroanalytical signal to an external computing device.

12. The device of claim 10 wherein the electrophysiological signal and/or the electroanalytical signal is an analogue signal.

13. The device of claim 10 wherein the processed electrophysiological signal and/or the processed electroanalytical signal is:
i. an amplified signal; and/or
ii. a digital signal.

14. The device of any one of the preceding claims, further comprising one or more of the following:
i. an accelerometer for the detection of maternal movement and/or the movement of a biosensor device;
ii. connection studs;
iii. an adhesive patch, strap, or bandage for securing the electronic processing unit to the subject if the subject is not a fetus (for example the subject is exercising);
iv. an adhesive patch or a strap or a bandage for securing the electronic processing unit to the mother of a fetus;
v. a top cover; and
vi. a bottom cover.

15. A system for processing an electroanalytical signal indicative of the concentration of an analyte in a subject and an electrophysiological signal of the subject, the system comprising:
a) a sensor device attachable to the subject, said sensor device comprising (i) a biosensor device capable of and operable for sensing the concentration of the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of the subject;
b) a transmission means to transmit the electroanalytical signal and the electrophysiological signal to a device for processing signals indicative of health; and
c) the device for processing signals indicative of health according to claim 1.

16. A method for processing an electroanalytical signal indicative of the concentration of an analyte in a subject and an electrophysiological signal of the subject, the method comprising the steps of:
a) attaching a sensor device to the subject, said sensor device comprising (i) a biosensor device capable of and operable for sensing the concentration of the analyte in the subject and (ii) an electrocardiogram (ECG) capable of and operable for sensing an ECG waveform of the subject, wherein said biosensor device of the sensor device generates an electroanalytical signal indicative of the concentration of the analyte in the subject and said ECG of the sensor device generates an electrophysiological signal of a subject;
b) transmitting the electroanalytical signal and the electrophysiological signal to a device for processing signals indicative of health via a transmission means; and
c) processing the electroanalytical signal and the electrophysiological signal in the device for processing signals indicative of health, the device for processing signals indicative of health according to claim 1.

17. The device of claim 2, system of claim 14 or method of claim 15, wherein the analyte is lactate.
